(19) United States
(12) Patent Application Publication
(10) Pub. No.: US 2024/0257949 A1
(43) Pub. Date: Aug. 1, 2024

(54) SYSTEMS AND METHODS FOR NOISE-AWARE SELF-SUPERVISED ENHANCEMENT OF IMAGES USING DEEP LEARNING

(71) Applicant: Subtle Medical, Inc., Menlo Park, CA (US)

(72) Inventors: Long WANG, Mountain View, CA (US); Gajanana Keshava DATTA, Sunnyvale, CA (US); Enhao GONG, Sunnyvale, CA (US)

(21) Appl. No.: 18/602,468

(22) Filed: Mar. 12, 2024

Related U.S. Application Data
(63) Continuation of application No. PCT/US2022/044714, filed on Sep. 26, 2022.
(60) Provisional application No. 63/249,929, filed on Sep. 29, 2021.

(51) Int. Cl. G16H 30/40; G06T 5/50; G06T 5/60

(52) U.S. Cl. CPC G16H 30/40; G06T 5/50; G06T 5/60; G06T 2207/20081; G06T 2207/20084

(57) ABSTRACT

Methods and systems are provided for improving quality of medical images. The deep learning method uses only noisy image for training, unlike the supervised methods that require pairs of noisy and ground truth images. By using the natural architecture search and exploring the search space, an improved network architecture is obtained for the enhancement tasks, which finds a balance between the noise distribution and the convolution features. The method provides the self-supervised samplers which utilize the correlation between the noise patterns and applies the dropout-enabled ensemble to further increase the enhancement effect.

### CROSS-REFERENCE TO RELATED APPLICATION

This application is a Continuation of International Application No. PCT/US2022/044714, filed Sep. 26, 2022, which claims priority to U.S. Provisional Application No. 63/249,929 filed on Sep. 29, 2021, the content of which is incorporated herein in its entirety.

### BACKGROUND

Contrast agents such as Gadolinium-based contrast agents (GBCAs) have been used in approximately one third of Magnetic Resonance imaging (MRI) exams worldwide to create indispensable image contrast for a wide range of clinical applications, but pose health risks for patients with renal failure and are known to deposit within the brain and body for patients with normal kidney function. Recently, deep learning technique has been used to reduce GBCA dose in volumetric contrast-enhanced MRI, but challenges in generalizability remain due to variability in scanner hardware and clinical protocols within and across sites.

While significant progress has been made in developing fast acquisition methods for magnetic resonance imaging (MRI), they may suffer from degraded image quality under challenging conditions (ultra-fast imaging, low fields, etc) or due to imaging artifacts. In this situation, generating a good image as the ground-truth image for training a supervised neural network is very challenging. For instance, either longer acquisition time or complicated reconstruction processes may be required to compensate for the poor image quality.

### SUMMARY

The present disclosure provides improved imaging systems and methods that can address various drawbacks of conventional systems, including those recognized above. Methods and systems as described herein can improve image quality with reduced dose level of contrast agent such as Gadolinium-Based Contrast Agents (GBCAs). In particular, the present disclosure provides a self-supervised based robust image enhancement system that can significantly improve the image quality of the initial degraded image to further accelerate the acquisition (e.g., allows for faster imaging). The low quality image herein may be referred to as degraded image which may comprise images acquired with reduced dose of contrast agent, accelerated acquisition, or acquired under standard conditions but degraded due to other reasons. Examples of low quality in medical imaging may include a variety of artifacts, such as noise (e.g., low signal noise ratio), blur (e.g., motion artifact), shading (e.g., blockage or interference with sensing), missing information (e.g., missing pixels or voxels in painting due to removal of information or masking), reconstruction (e.g., degradation in the measurement domain), and/or under-sampling artifacts (e.g., under-sampling due to compressed sensing, aliasing). The image quality enhancement methods and systems herein may beneficially improve images with various artifacts or various noise distributions. In particular, the present disclosure provides systems and methods may be capable of generating an image with higher image quality and/or generating training pairs for enhancement and train efficient supervised models for specific purposes.

In an aspect, a computer-implemented method is for improving image quality. The method comprises: (a) identifying architecture for a deep learning network model based at least in part on an original medical image of a subject, where the original medical image has a low quality; (b) generating a pair of images of low-quality from the original medical image; (c) training the deep learning network model based on the pair of images of low-quality, wherein the deep learning network model has the architecture identified in (a); and (d) making inference with deep learning network model to output an enhanced medical image with dropout enabled.

In a related yet separate aspect, a non-transitory computer-readable storage medium including instructions that, when executed by one or more processors, cause the one or more processors to perform operations. The operations comprise: (a) identifying architecture for a deep learning network model based at least in part on an original medical image of a subject, where the original medical image has a low quality; (b) generating a pair of images of low-quality from the original medical image; (c) training the deep learning network model based on the pair of images of low-quality, wherein the deep learning network model has the architecture identified in (a); and (d) making inference with deep learning network model to output an enhanced medical image with dropout enabled.

In some embodiments, the medical image is acquired using a medical imaging apparatus with shortened scanning time or reduced amount of tracer dose. In some embodiments, the architecture for the deep learning network model is identified by employing a natural architecture search algorithm. In some cases, the natural architecture search algorithm comprises reinforcement learning with a recurrent neural network controller.

In some embodiments, the pair of images of low-quality is generated using a sampler method. In some cases, the sampler method is selected from a group consisting of self2self sampler method and neigher2neighbor sampler method. In some cases, the sampler method is selected based at least in part on a noise distribution in the medical image.

In some embodiments, the training dataset for training the deep learning network model includes medical images of low quality only. In some embodiments, the deep learning network model is trained using self-supervised learning. In some embodiments, the enhanced medical image is an average of multiple inferences made by the deep learning network model by dropping nodes in the deep learning network model randomly.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and descriptions are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an exemplary diagram for the network architecture searching process.

FIG. 2 shows different examples of sampler methods.

FIG. 3 shows an example of a self2self sampling-based training scheme.

FIG. 4 shows an example of a Neighbor2neighbor sampling-based training scheme.

FIG. 5 shows an example of the ensemble inference scheme.

FIG. 6A and FIG. 6B shows example of results generated by the methods herein.

FIG. 7 shows an example of a computing platform implementing the methods and systems consistent with those described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure provides improved imaging systems and methods that can address various drawbacks of conventional systems, including those recognized above. Methods and systems as described herein can improve image quality with reduced dose level of contrast agent such as Gadolinium-Based Contrast Agents (GBCAs). In particular, the present disclosure provides a self-supervised-based robust image enhancement system that can significantly improve the image quality of the initial degraded image to further accelerate the acquisition (e.g., allows for faster imaging). The image quality enhancement methods and systems herein may beneficially improve images with various artifacts or various noise distributions. In particular, the present disclosure provides systems and methods may be capable of generating an image with higher image quality. In some embodiments, methods and systems herein may be capable of generating training pairs for enhancement without requiring labeled data and training efficient supervised models for specific purposes with the generated training pairs.

Though MR image, denoising examples are primarily provided herein, it should be understood that the present approach, models, methods and systems may be used in other imaging modality contexts or other image restoration tasks. For instance, the presently described approach may be employed on data acquired by other types of tomographic scanners including, but not limited to, positron emission tomography (PET), computed tomography (CT), single photon emission computed tomography (SPECT) scanners, functional magnetic resonance imaging (fMRI) scanners and the like. Methods, systems and/or components of the systems or models may be used in other image restoration tasks (e.g., super-resolution, image denoising, image inpainting, and image dehazing, and image-to-image translation etc.).

The term low quality image as utilized herein may refer to degraded image which may comprise images acquired with reduced dose of contrast agent, accelerated acquisition, or acquired under standard conditions but degraded due to other reasons. Examples of low quality in medical imaging may include a variety of artifacts, such as noise (e.g., low signal noise ratio), blur (e.g., motion artifact), shading (e.g., blockage or interference with sensing), missing information (e.g., missing pixels or voxels in painting due to removal of information or masking), reconstruction (e.g., degradation in the measurement domain), under-sampling artifacts (e.g., under-sampling due to compressed sensing, aliasing), and/or other artifacts (e.g., image corruption).

In some embodiments, the system and method herein may be capable of generating high-quality images using only low-quality images (e.g., corrupted images, noisy images, etc) for training the deep learning model. In some embodiments, the method may comprise: a) conducting a natural architecture search to identify the network that is suitable for a given or the current noise distribution, b) applying one or more sampling methods to generate the training pairs from the low quality (e.g., noisy) input image only and training the network c) enabling the dropout layer, generating inference results, and running an ensemble during the inference phase.

The method herein provides multiple advantages over existing methods. For example, unlike the supervised methods that require pairs of low quality (e.g., noisy) and high quality image i.e., ground truth images, the methods herein may include provide a self-supervised enhancement method requiring only the low quality image (e.g., noisy image) for training. Compared to the current image denoising method such as methods based on block matching with 4D filtering (BM4D), the method herein can provide improved performance and preserve the small structures (e.g., fine features or high frequency features) well without introducing additional artifacts.

In some embodiments, the methods and systems herein may provide an improved network architecture for the image quality enhancement tasks utilizing the natural architecture search and exploring the search space. In some cases, the network architecture may be improved by taking into account both the noise distribution and the convolution features. For instance, the method herein may provide a self-supervised enhancement method utilizing the correlation between the noise distribution and convolution features (e.g., kernel size, convolutional layer size, etc.) as well as the natural architecture search to improve the performance of the image quality enhancement tasks.

In some embodiments, methods and systems herein may further improve the performance by employing the drop out layer during inference. Current methods may not use the dropout layer in the regression tasks and the drop out layers are defaulted to be disabled during the inference phase. The methods herein may enable the dropout layer during the inference phase and beneficially reduce the variance of the prediction by generating multiple inference results with the dropped out node.

### Preprocessing

In some embodiments, the methods herein may comprise preprocessing the data. In some cases, the preprocessing method may comprise, for example, applying max normalization, threshold clipping, and/or interpolation to the same image scale during preprocessing.

### Natural Architecture Search

Different network architectures (e.g., the convolution features such as kernel size, convolution size, etc.) may yield a significant difference for denoising effects for images with various noise distributions. The methods herein may beneficially identify an appropriate network architecture for processing a given type image quality such as noise distribution.

In some embodiments, the methods herein may provide an improved natural architecture search (NAS) algorithm that is capable of identifying network architecture (e.g., encoder, decoder design, etc.) for a U-Net. A U-Net may concatenate the encoded features at the corresponding encoder layer with the features in the decoder when performing a sequence of up-convolutions. In a conventional U-Net network, the architectures such as skip connection patterns may be manually designed and fixed for each task.

The methods herein may employ natural architecture search (NAS) algorithm (NAS) algorithm in discovering networks with an optimal performance in a large search space. In some cases, the algorithm may leverage reinforcement learning (RL) with a recurrent neural network (RNN) controller and use the Peak Signal to Noise Ratio (PSNR) as the reward to guide the architecture search.

In some embodiments, the provided NAS algorithm may extend the search space. In some cases. the search space may be extended to a plurality of steps or operations including, for example, spatial upsampling methods (e.g., bilinear, bicubic, nearest-neighbor, depth-to-space, stride 2 transposed convolution, etc.), operations for feature transformation (e.g., 2D convolution, add every N consecutive channels, separable convolution, depthwise convolution), kernel size (e.g., 1×1, 3×3, 5×5, 7×7, etc.), dilation rate (e.g., 1, 3, 5, 7), activation (e.g., none, ReLU, LeakyReLtU, SELU, PReLU). It should be noted that the above mentioned search space for changing the spatial resolution of the feature map (i.e., spatial upsampling methods), methods of feature transformation and the like the for illustration purpose only, the options or methods may include any newly developed methods (e.g., spatial upsampling operators CARAFE).

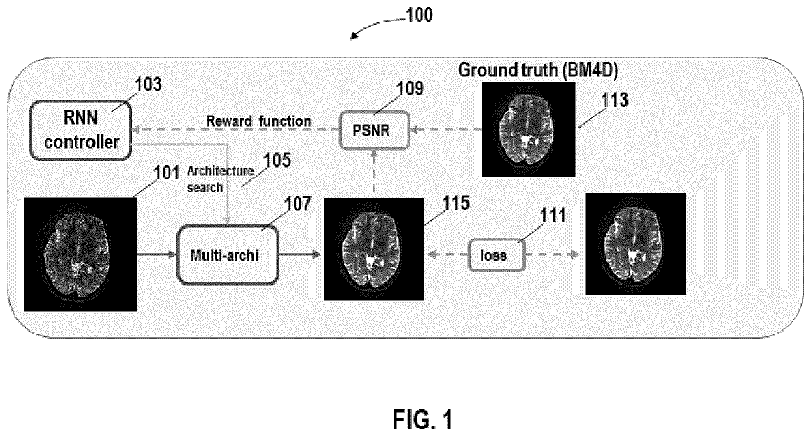

In some cases, a network architecture may be identified based at least in part on a given degraded input image. FIG. 1 shows an exemplary diagram for the NAS searching process 100. During the searching 100 process, the network takes a low quality image (e.g., noisy image 101) as input and the BM4D processed image as ground truth 113, and uses an RNN controller 103 and PSNR 109 as the reward function to search for the best performing network architecture 105. The NAS algorithm may comprise applying reinforcement learning with an RNN controller using the PSNR as the reward to search for the best-performing network structure on a held-out training set. The best performance network structure may be identified by ranking each of the searched network structure 107 by computing the Peak Signal to Noise Ratio (PSNR) between the ground-truth image 113 and the network's output 115 and determine the best-performing network structure for the training set.

After the network architecture search, the architecture (e.g., best performing architecture) may be transferred for training the model. In some embodiments, self-supervised training algorithm may be employed for training the model. The self-supervised training framework may comprise a training image pairs generation method based on sub-sampling and a self-supervised training scheme with a regularization term. In some cases, the self-supervised training framework may comprise generating training pairs from the original degraded image using different sampling methods. The self-supervised training framework may be capable of training the enhancement network with only single degraded image (i.e., single low quality image without paired high-quality image). In some cases, based on the various degraded distributions (eg. noise distributions), different resampling methods (e.g., self2self or neighbor2neighbor) may be applied to generate training pairs from the original low quality (e.g., noisy) image.

### Sampling Methods

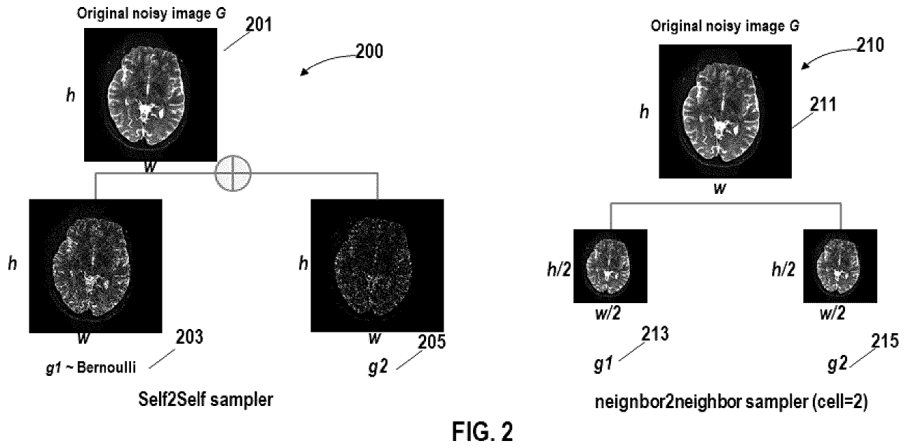

Based on the various degraded distributions, different resampling methods may be selected and applied to generate the training pairs from the original noisy image. FIG. 2 shows different examples of the sampler methods 200, 210.

The different sampler methods may include at least a self2self sampler method 200 and a neighbor2neighbor sampler method 210. In the illustrated example of the self2self sampler 200, a binary Bernoulli vector b is sampled from a Bernoulli distribution with probability p(0,1) on the image space. Two sets of sub images (g₁, g₂) (e.g., image pairs 203, 205) may be generated from the original degraded image G 201 and can be utilized as training pairs for training a model in a self-supervised learning process. The image pairs (g₁, g₂) may be noisy defined as follows:

$${g}_{1} := {b}_{m} ⊙ G$$

$${g}_{2} := ( 1 - {b}_{m} ) ⊙ G$$

where G is the original noisy image, bₘ is the binary Bernoulli vector.

In some cases, the neighbor2neighbor sampler method 210 may use random neighbor sub-samplers to generate a noisy image pairs (g₁, g₂) (e.g., image pairs 213, 215) from a single noisy images G 201 for training the model. For example, an image may be divided into w/k×w/k cells with size 2×2. In each 2×2 cell, two neighboring pixels may be randomly chosen.

Various sampler methods may be selected to take a single noisy image as input and generate the training pairs (e.g., image pairs of noisy images) for the training process. However, different sampler methods may lead to different sampler result (e.g., the sub-sampled image pairs may be blurred out). In some embodiments, the methods herein may select an appropriate sampler method from a plurality of sampler methods based on the noise distribution in the original input image. In some cases, the sampler method may be selected in real-time from the plurality of sampler methods based on the real-time data. For instance, by applying the plurality of sampler methods to the same original input noisy image, one sampler method may be selected based on a comparison of the results (e.g., not blur out the image). Additionally or alternatively, the sampler method may be selected based on empirical data. For instance, based on empirical data, the self2self method may be suitable for the image with global distorted features and the neighbor2neighbor method may be suitable with the images with local distorted features. A sampler method may be selected by initially determining a type of the degradation or artifacts in the original image (e.g., global distorted features or local distorted features), then a sampler method may be selected based on the empirical data.

### Training Scheme

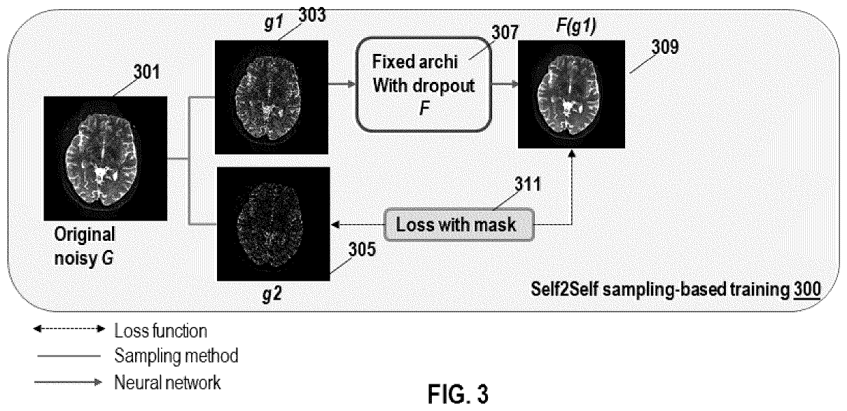

The network architecture transferred from the NAS phase and the training pairs generated as described above may then be utilized in a self-supervised training process. In the cases, the training framework may be different based on the noisy image pairs generated by the selected sampler method. Alternatively, the training framework may be the same for training image pairs generated by the different sampler methods. FIG. 3 shows an example of a self2self sampling-based training scheme 300. The training image pairs 303, 305 may be generated from the original low quality image 301 using a sampler method as described above. In the illustrated example, the training pair (g₁, g₂) (e.g., image pairs 303, 305) may be Bernoulli sampled using the self2self sampler method as described above and the Bernoulli sampled image pairs 303, 305 may be used to train a deep neural network (NN) 307, denoted by Fθ(·) with the parameter vector θ, that maps a low-quality (e.g., noisy image) to its high quality (clean) counterpart image.

The deep neural network 307 may be trained by minimizing a loss function. In particular, the training of the NN may use dropout (e.g., Bernoulli dropout) to reduce the variance of the prediction. An example of the loss function 311 is Li norm loss as the following:

$$min {\sum }_{m = 1}^{M} {‖ {F}_{Θ} ( {g}_{1} ) - {g}_{2} ‖}_{{b}_{m}}$$

wherein bₘ is the binary Bernoulli vector whose entries are independently sampled from a Bernoulli distribution with probability p ∈ (0, 1). The NN 307 is based on the architecture with the best performance identified by the NAS algorithm as described above. In the illustrated example, the loss of each pair (prediction 309 and sampled image 305) is measured only on those pixels that are masked by bₘ. As the masked pixels are randomly selected using a Bernoulli process, the summation of the loss over all pairs measures the difference over all image pixels. With the above training scheme with the Bernoulli sampling, the convergence of the NN to an identity mapping can be avoided.

In some embodiments. instead of using the Bernoulli dropout as the regularization for the deep NN, the framework may use an additional regularization term in the loss function. In some embodiments, when the sampling method is the neighbor2neighbor sampler, the loss function may be derived from two aspects, one is from the reconstruction loss, another is from the regularizer loss.

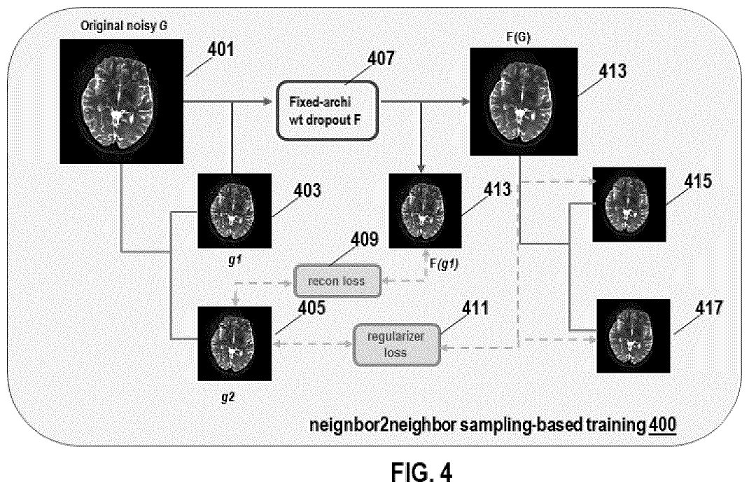

FIG. 4 shows an example of the Neighbor2neighbor sampling-based training scheme 400. The loss function may include a regularization term to address the essential difference of pixel ground-truth values between neighbors on the original noisy image. As an example, the loss function is the following:

$$L = {L}_{rec} + γ {L}_{reg} = {‖ {f}_{θ} ( {g}_{1} ( y ) ) - {g}_{2} ( y ) ‖}_{2}^{2} + γ {‖ {f}_{θ} ( {g}_{1} ( y ) ) - {g}_{2} ( y ) - ( {g}_{1} ( {f}_{θ} ( y ) ) - {g}_{2} ( {f}_{θ} ( y ) ) ) ‖}_{2}^{2}$$

g₁(y) and g₂(y)are the input and output for the network, ƒ₇₄ (g₁(y)) is the predicted result, y is the original noisy image, y is a hyper-parameter controlling the strength of the regularization term. As illustrated in the example, a Neighbor2neighbor sampler may be applied to an original degraded image 401 to generate a pair of degraded images (e.g., noisy image pair g1 403 and g2 405). The deep NN 407 may have the architecture identified by the NAS algorithm as described above and the parameters may be optimized by minimizing the loss function comprising the reconstruction loss 409 and the regularizer loss 411. The reconstruction loss is a function of the predicted result 413 and the generated sampler image g2 405. The regularizer loss 411 includes the essential difference of the ground-truth pixel values (ground truth image pairs 415, 417) between the sub-sampled noisy image pair.

### Ensemble

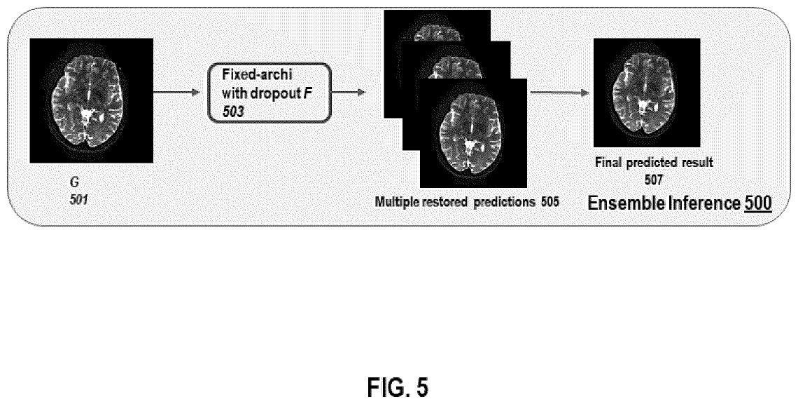

As described above, the methods herein may reduce the variance of an NN predictions by utilizing the dropout in both the training and inference stage. In particular, the methods herein may comprise dropout-based ensemble. FIG. 5 shows an example of the ensemble inference scheme 500. Dropout layer is normally enabled at the training process and disabled at the inference process. During training process, dropout is regularization technique for deep NNs.

Dropout may refer to randomly dropping out nodes when training an NN, which can be viewed as using a single NN to approximate a large number of different NNs. Dropout provides a computationally-efficient way to train and maintain multiple NN models for prediction.

However, owing to model uncertainty introduced by dropout, the predictions from these models are likely to have certain degree of statistical independence leading to the variance in the prediction result.

In the provided ensemble inference process 500, the dropout layer may be enabled to improve the image quality (e.g., sharpness). As described above, during the training stage, the dropout layers may be enabled and for the multiple training pairs, multiple predictions may be generated.

During the inference of the image quality enhancement (e.g., denoising), the dropout lay ers may be enabled to form multiple NNs. For example, multiple NNs are formed by running dropout on the configured layers of the trained NN 503. Then, multiple restored images 505 are generated by feeding an input image 501 to each of the newly-formed NNs 503. The multiple restored images 505 may be averaged to generate the final result 507.

In some cases, inference with dropout-based ensemble may comprise dropping nodes randomly in the neural network (NN) to generate multiple inferences/predictions and the final output may be the average of the multiple predictions. In some cases, inference with dropout-based ensemble may comprise both dropping nodes in the NN and dropping pixels (Bernoulli sampling) in the input noisy image. In some cases, the input 501 for self2self sampler is the Bernoulli sampled image g1. In some cases, the input 501 for neighbor2neighbor sampler method is the original degraded image.

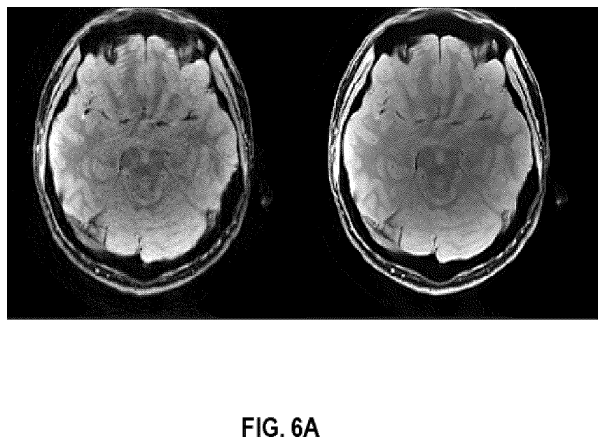

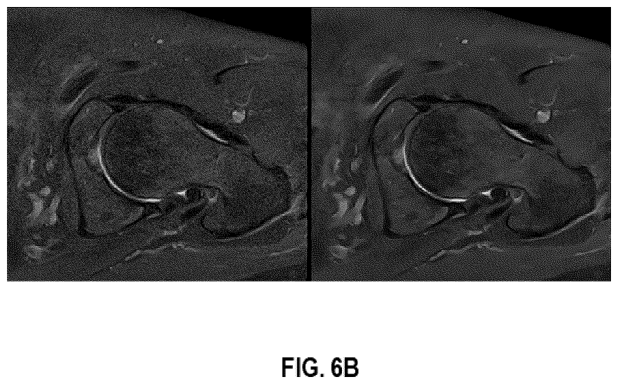

FIG. 6A shows example of results from self2self self-supervised method. FIG. 6B shows example of results from neighbor2neighbor method. As illustrated in the examples, the quality of the image has been significantly enhanced using the provided methods.

### System Overview

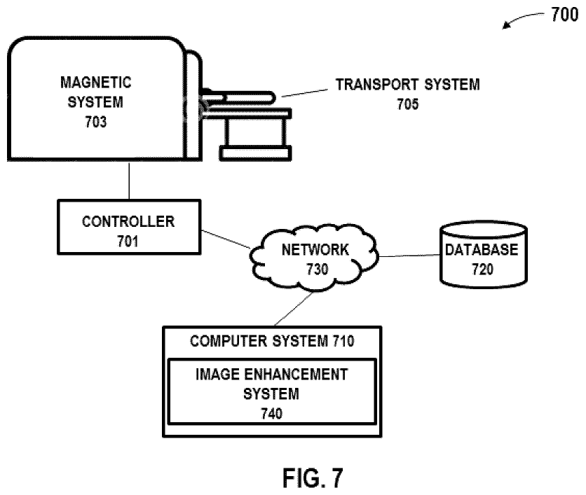

The systems and methods can be implemented on existing imaging systems such as but not limited to MR imaging systems or various other imaging modalities without a need of a change of hardware infrastructure. Alternatively, the systems and methods can be implemented by any computing systems that may not be coupled to the MR imaging system. For instance, methods and systems herein may be implemented in a remote system, one or more computer servers, which can enable distributed computing, such as cloud computing. FIG. 7 schematically illustrates an example MR system 700 comprising a computer system 710 and one or more databases operably coupled to a controller over the network 730. The computer system 710 may be used for further implementing the methods and systems as described for processing the medical images (MR images) for image quality enhancement (e.g., denoising, increase sharpness, contrast, etc.).

The controller 701 may be operated to provide the MRI sequence controller information about a pulse sequence and/or to manage the operations of the entire system, according to installed software programs. The controller may also serve as an element for instructing a patient to perform tasks, such as, for example, a breath hold by a voice message produced using an automatic voice synthesis technique. The controller may receive commands from an operator which indicate the scan sequence to be performed. The controller may comprise various components such as a pulse generator module which is configured to operate the system components to carry out the desired scan sequence, producing data that indicate the timing, strength and shape of the RF pulses to be produced, and the timing of and length of the data acquisition window. Pulse generator module may be coupled to a set of gradient amplifiers to control the timing and shape of the gradient pulses to be produced during the scan. Pulse generator module also receives patient data from a physiological acquisition controller that receives signals from sensors attached to the patient, such as ECG (electrocardiogram) signals from electrodes or respiratory signals from a bellows. Pulse generator module may be coupled to a scan room interface circuit which receives signals from various sensors associated with the condition of the patient and the magnet system. A patient positioning system may receive commands through the scan room interface circuit to move the patient to the desired position for the scan.

The controller 701 may comprise a transceiver module which is configured to produce pulses which are amplified by an RF amplifier and coupled to RF coil by a transmit/receive switch. The resulting signals radiated by the excited nuclei in the patient may be sensed by the same RF coil and coupled through transmit/receive switch to a preamplifier. The amplified nuclear magnetic resonance (NMR) signals are demodulated, filtered, and digitized in the receiver section of transceiver. Transmit/receive switch is controlled by a signal from pulse generator module to electrically couple RF amplifier to coil for the transmit mode and to preamplifier for the receive mode. Transmit/receive switch may also enable a separate RF coil (for example, a head coil or surface coil, not shown) to be used in either the transmit mode or receive mode.

The NMR signals picked up by RF coil may be digitized by the transceiver module and transferred to a memory module coupled to the controller. The receiver in the transceiver module may preserve the phase of the acquired NMR signals in addition to signal magnitude. The down converted NMR signal is applied to an analog-to-digital (A/D) converter (not shown) which samples and digitizes the analog NMR signal. The samples may be applied to a digital detector and signal processor which produces in-phase (I) values and quadrature (Q) values corresponding to the received NMR signal. The resulting stream of digitized I and Q values of the received NMR signal may then be employed to reconstruct an image. The provided methods herein may take the reconstructed image as input and process for detection and classification purpose.

The controller 701 may comprise or be coupled to an operator console (not shown) which can include input devices (e.g., keyboard) and control panel and a display. For example, the controller may have input/output (I/O) ports connected to an I/O device such as a display, keyboard and printer. In some cases, the operator console may communicate through the network with the computer system 710 that enables an operator to control the production and display of images on a screen of display.

The system 700 may comprise a user interface. The user interface may be configured to receive user input and output information to a user. The user input may be related to control of image acquisition. The user input may be related to the operation of the MRI system (e.g., certain threshold settings for controlling program execution, parameters for controlling the joint estimation of coil sensitivity and image reconstruction, etc). The user input may be related to various operations or settings about the detection and classification system 740. The user input may include, for example, a selection of a target structure or ROI, training parameters, setting an image acceleration parameter, displaying settings of a reconstructed image, customizable display preferences, selection of an acquisition scheme, and various others. The user interface may include a screen such as a touch screen and any other user interactive external device such as handheld controller, mouse, joystick, keyboard, trackball, touchpad, button, verbal commands, gesture-recognition, attitude sensor, thermal sensor, touch-capacitive sensors, foot switch, or any other device.

The MRI platform 700 may comprise computer systems 710 and database systems 720, which may interact with the controller. The computer system can comprise a laptop computer, a desktop computer, a central server, distributed computing system, etc. The processor may be a hardware processor such as a central processing unit (CPU), a graphic processing unit (GPU), a general-purpose processing unit, which can be a single core or multi core processor, a plurality of processors for parallel processing, in the form of fine-grained spatial architectures such as a field programmable gate array (FPGA), an application-specific integrated circuit (ASIC), and/or one or more Advanced RISC Machine (ARM) processors. The processor can be any suitable integrated circuits, such as computing platforms or microprocessors, logic devices and the like. Although the disclosure is described with reference to a processor, other types of integrated circuits and logic devices are also applicable. The processors or machines may not be limited by the data operation capabilities. The processors or machines may perform 512 bit, 256 bit, 128 bit, 64 bit, 32 bit, or 16 bit data operations.

The imaging platform 700 may comprise one or more databases. The one or more databases 720 may utilize any suitable database techniques. For instance, structured query language (SQL) or “NoSQL” database may be utilized for storing image data, raw collected data, reconstructed image data, training datasets, validation dataset, trained model (e.g., hyper parameters), weighting coefficients, etc. Some of the databases may be implemented using various standard data-structures, such as an array, hash, (linked) list, struct, structured text file (e.g., XML), table, JSON, NOSQL and/or the like. Such data-structures may be stored in memory and/or in (structured) files. In another alternative, an object-oriented database may be used. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. If the database of the present disclosure is implemented as a data-structure, the use of the database of the present disclosure may be integrated into another component such as the component of the present disclosure. Also, the database may be implemented as a mix of data structures, objects, and relational structures. Databases may be consolidated and/or distributed in variations through standard data processing techniques. Portions of databases, e.g., tables, may be exported and/or imported and thus decentralized and/or integrated.

The network 730 may establish connections among the components in the imaging platform and a connection of the imaging system to external systems. The network 730 may comprise any combination of local area and/or wide area networks using both wireless and/or wired communication systems. For example, the network 730 may include the Internet, as well as mobile telephone networks. In one embodiment, the network 730 uses standard communications technologies and/or protocols. Hence, the network 730 may include links using technologies such as Ethernet, 802.11, worldwide interoperability for microwave access (WiMAX), 2G/3G/4G mobile communications protocols, asynchronous transfer mode (ATM), InfiniBand, PCI Express Advanced Switching, etc. Other networking protocols used on the network 230 can include multiprotocol label switching (MPLS), the transmission control protocol/Internet protocol (TCP/IP), the User Datagram Protocol (UDP), the hypertext transport protocol (HTTP), the simple mail transfer protocol (SMTP), the file transfer protocol (FTP), and the like. The data exchanged over the network can be represented using technologies and/or formats including image data in binary form (e.g., Portable Networks Graphics (PNG)), the hypertext markup language (HTML), the extensible markup language (XML), etc. In addition, all or some of links can be encrypted using conventional encryption technologies such as secure sockets layers (SSL), transport layer security (TLS), Internet Protocol security (IPsec), etc. In another embodiment, the entities on the network can use custom and/or dedicated data communications technologies instead of, or in addition to, the ones described above.

Systems and methods of the present disclosure may provide an image enhancement system 740 that can be implemented in software, hardware, firmware, embedded hardware, standalone hardware, application specific-hardware, or any combination of these. The image enhancement system 740 can be a standalone system that is separate from the MR imaging system. The image enhancement system 740 may be in communication with the MR imaging system such as a component of a controller of the MR imaging system. In some embodiments, the detection and classification system 740 may comprise multiple components, including but not limited to, a training module, an image enhancement module and a user interface module.

The training module may be configured to train the image enhancement model framework as described above. For instance, the training module may be configured to conduct a natural architecture search to identify the network that is suitable for a given or the current noise distribution, apply different sampling methods to generate the training pairs from the low quality (e.g., noisy) input image only and training the network, and train a deep neural network using self-supervised learning enabling the dropout layer.

The training module may be configured to obtain and manage training datasets. For example, the training datasets for the detection network may comprise low-quality or degraded MR images from a subject. In some cases, the training datasets may comprise low-quality image only. The training module may be configured to train the enhancement network as described elsewhere herein. The training module may train a model off-line. Alternatively or additionally, the training module may use real-time data as feedback to refine the model for improvement or continual training.

The enhancement module may be configured to perform image quality enhancement (e.g., denoising) using trained models obtained from the training module. The enhancement module may deploy and implement the trained model for making inferences, and run an ensemble during the inference phase with the dropout layers enabled.

The user interface module may permit users to view the training result, view predicted results or interact with the training process.

Methods as described herein can be implemented by way of machine (e.g., computer processor) executable code stored on an electronic storage location of the computer system 710, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein A and/or B encompasses one or more of A or B, and combinations thereof such as A and B. It will be understood that although the terms “first,” “second,” “third” etc. are used herein to describe various elements, components, regions and/or sections, these elements, components, regions and/or sections should not be limited by these terms. These terms are merely used to distinguish one element, component, region or section from another element, component, region or section. Thus, a first element, component, region or section discussed herein could be termed a second element, component, region or section without departing from the teachings of the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including,” when used in this specification, specify the presence of stated features, regions, integers, steps, operations, elements and/or components, but do not preclude the presence or addition of one or more other features, regions, integers, steps, operations, elements, components and/or groups thereof.

Reference throughout this specification to “some embodiments,” or “an embodiment,” means that a particular feature, structure, or characteristic described in connection with the embodiment is included in at least one embodiment. Thus, the appearances of the phrase “in some embodiment,” or “in an embodiment,” in various places throughout this specification are not necessarily all referring to the same embodiment. Furthermore, the particular features, structures, or characteristics may be combined in any suitable manner in one or more embodiments.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A computer-implemented method for improving image quality comprising:
(a) identifying architecture for a deep learning network model based at least in part on an original medical image of a subject, wherein the original medical image has a low quality;
(b) generating a pair of images of low-quality from the original medical image;
(c) training the deep learning network model based on the pair of images of low-quality, wherein the deep learning network model has the architecture identified in (a); and
(d) making inference with deep learning network model to output an enhanced medical image with dropout enabled.

2. The computer-implemented method of claim 1, wherein the medical image is acquired using a medical imaging apparatus with shortened scanning time or reduced amount of tracer dose.

3. The computer-implemented method of claim 1, wherein the architecture for the deep learning network model is identified by employing a natural architecture search algorithm.

4. The computer-implemented method of claim 3, wherein the natural architecture search algorithm comprises reinforcement learning with a recurrent neural network controller.

5. The computer-implemented method of claim 1, wherein the pair of images of low-quality is generated using a sampler method.

6. The computer-implemented method of claim 5, wherein the sampler method is selected from a group consisting of self2self sampler method and neigher2neighbor sampler method.

7. The computer-implemented method of claim 5, wherein the sampler method is selected based at least in part on a noise distribution in the medical image.

8. The computer-implemented method of claim 1, wherein the training dataset for training the deep learning network model includes medical images of low quality only.

9. The computer-implemented method of claim 1, wherein the deep learning network model is trained using self-supervised learning.

10. The computer-implemented method of claim 1, wherein the enhanced medical image is an average of multiple inferences made by the deep learning network model by dropping nodes in the deep learning network model randomly.

11. A non-transitory computer-readable storage medium including instructions that, when executed by one or more processors, cause the one or more processors to perform operations comprising:
(a) identifying architecture for a deep learning network model based at least in part on an original medical image of a subject, wherein the original medical image has a low quality;
(b) generating a pair of images of low-quality from the original medical image;
(c) training the deep learning network model based on the pair of images of low-quality, wherein the deep learning network model has the architecture identified in (a); and
(d) making inference with deep learning network model to output an enhanced medical image with dropout enabled.

12. The non-transitory computer-readable storage medium of claim 11, wherein the medical image is acquired using a medical imaging apparatus with shortened scanning time or reduced amount of tracer dose.

13. The non-transitory computer-readable storage medium of claim 11, wherein the architecture for the deep learning network model is identified by employing a natural architecture search algorithm.

14. The non-transitory computer-readable storage medium of claim 13, wherein the natural architecture search algorithm comprises reinforcement learning with a recurrent neural network controller.

15. The non-transitory computer-readable storage medium of claim 11, wherein the pair of images of low-quality is generated using a sampler method.

16. The non-transitory computer-readable storage medium of claim 15, wherein the sampler method is selected from a group consisting of self2self sampler method and neigher2neighbor sampler method.

17. The non-transitory computer-readable storage medium of claim 15, wherein the sampler method is selected based at least in part on a noise distribution in the medical image.

18. The non-transitory computer-readable storage medium of claim 11, wherein the training dataset for training the deep learning network model includes medical images of low quality only.

19. The non-transitory computer-readable storage medium of claim 11, wherein the deep learning network model is trained using self-supervised learning.

20. The non-transitory computer-readable storage medium of claim 11, wherein the enhanced medical image is an average of multiple inferences made by the deep learning network model by dropping nodes in the deep learning network model randomly.
